(19) United States
(12) Patent Application Publication
(10) Pub. No.: US 2026/0007123 A1
(43) Pub. Date: Jan. 8, 2026

(54) THYMOCYTE HUMANIZED ANIMALS, METHODS OF MAKING AND METHODS OF USE THEREOF

(71) Applicant: Wisconsin Alumni Research Foundation, Madison, WI (US)

(72) Inventor: Matthew Brown, Madison, WI (US)

(73) Assignee: Wisconsin Alumni Research Foundation, Madison, WI (US)

(21) Appl. No.: 19/258,282

(22) Filed: Jul. 2, 2025

Related U.S. Application Data
(60) Provisional application No. 63/668,584, filed on Jul. 8, 2024.

(51) Int. Cl. A01K 67/0271

(52) U.S. Cl. CPC A01K 67/0271; A01K 2207/12; A01K 2207/15; A01K 2227/105; A01K 2227/108

(57) ABSTRACT

In an aspect, a method of making a humanized animal includes providing an immune-deficient animal lacking mature T cells, B cells and NK cells; and injecting a population of human thymocytes into the immune-deficient animal to provide the humanized animal; wherein the population of human thymocytes are thymocytes collected from human thymus or thymus tissue; and wherein the humanized animal includes mature human T cells expressing human CD8 and human CD4. In another aspect, also included is a humanized animal prepared by the foregoing method.

### CROSS-REFERENCE TO RELATED APPLICATIONS

This application claims priority to U.S. Provisional Application 63/668,584 filed on Jul. 8, 2024, which is incorporated herein by reference in its entirety.

### STATEMENT REGARDING FEDERALLY SPONSORED RESEARCH & DEVELOPMENT

This invention was made with government support under 75N93021C00004 awarded by the National Institutes of Health. The government has certain rights in the invention.

### FIELD OF THE DISCLOSURE

The present disclosure is related to methods of making humanized animals, and the humanized animals produced from the methods.

### BACKGROUND

Humanized rodent models have become a useful tool for pre-clinical experimental study of human immune function. Humanized rodent models are created by engrafting human cells and/or tissues into immunodeficient rodent strains. Mice engrafted with a human immune system are also referred to as human immune system mice (HIS-mice). (See, Ye and Chen, “Potential Applications and Perspectives of Humanized Mouse Models”, Annu. Rev. Anim. Biosci., 10:395-417 (2022); Shultz et al., “Humanized Mice for immune system investigation: progress, promise and challenges”, Nature Reviews: Immunology, 12:786-798 (2012)).

One of the earliest established mouse models is the human peripheral blood lymphocyte (PBL)-engrafted SCID mouse model (Hu-PBL-SCID), which are human PBL-engrafted severe combined immunodeficiency (SCID) mice, in which PBLs are delivered to adult recipient mice via injection such as intravenous, intraperitoneal, or intrasplenic injection. An advantage of Hu-PBL-SCID mice is the excellent engraftment of T cells. A disadvantage of Hu-PBL-SCID mice is the limited time frame for experimentation, thought to be due to graft versus host disease. Another disadvantage is the cost of the PBLs needed to create Hu-PBL-SCID mice.

Previously, a NeoThy mouse model was created by implanting non-fetal human thymus tissue from neonatal cardiac surgery patients into the kidney capsule. (Brown et al., “A Humanized Mouse Model Generated Using Surplus Neonatal Tissue”, Stem Cell Reports, 10:1175-1183 (2018); DOI: 10.1016/jstemcr.2018.02.011). Implantation of the thymus tissue was accompanied by injection of CD34-enriched hematopoietic stem cells (HSCs). The NeoThy mice exhibited robust engraftment of human T cells as well as other human immune cells including human CD45⁺ cells, CD19⁺ cells, CD3⁺ cells, B cells, and myeloid cells relevant for antigen presentation. While NeoThy mice are an important model for induced pluripotent stem cell immunogenicity studies, for example, the preparation process requires surgery/recovery.

What is needed are alternative strategies for producing humanized mouse models.

### BRIEF SUMMARY

In an aspect, a method of making a humanized animal comprises providing an immune-deficient animal lacking mature animal T cells, B cells and NK cells; and injecting a population of human thymocytes into the immune-deficient animal to provide the humanized animal; wherein the population of human thymocytes are thymocytes collected from human thymus or thymus tissue; and wherein the humanized animal comprises mature human T cells expressing human CD8 and human CD4.

In another aspect, also included is a humanized animal prepared by the above-described method.

### BRIEF DESCRIPTION OF THE DRAWINGS

FIG. 1 is a schematic of an embodiment of the production of a thymocyte humanized (Thymocyte-Hu) mouse according to the present disclosure.

FIG. 2 shows the input thymocyte CD4 and CD8 profile.

FIG. 3 illustrates the human chimerism of the mice over time.

FIG. 4 illustrates the survival of the Trisomy 2 (T21) and Diploid 21 (D21) mice. D21 mice are Thymocyte-Hu mice having 2 copies of chromosome 21, while T21 Thymocyte-Hu mice have 3 copies of chromosome 21.

FIG. 5 shows the characterization of the D21 and T21 Thymocyte-Hu mice.

FIG. 6 shows a Down syndrome (DS) mosaic iPSC line was differentiated into cardiomyocytes.

The above-described and other features will be appreciated and understood by those skilled in the art from the following detailed description, drawings, and appended claims.

### DETAILED DESCRIPTION

The inventor recognized that during the production of humanized NeoThy mice, for example, excess thymocytes are left over during processing of thymus tissue. In addition, the thymus and thymocytes can be a waste product of common clinical procedures. Injection of human thymocytes was thus explored as an alternative to implanting human thymus tissue to produce humanized mice.

More specifically, about 10 million or more human thymocytes collected from the thymus of neonatal heart patients were injected into the tail of immune-deficient mice. The thymocyte-injected mice maintain chimerism and live longer than PBL-Hu mice, for example. These mice are referred to herein as Thymocyte-Hu mice.

A thymocyte is a lymphocyte found in the thymus. Thymocytes are produced as stem cells in the bone marrow and reach the thymus via the bloodstream. Thymocytes mature into T cells via a process called thymopoiesis. Thymocytes can be isolated during processing of neonatal thymus tissues, for example, and are distinct from the neonatal thymus tissue used to produce NeoThy mice.

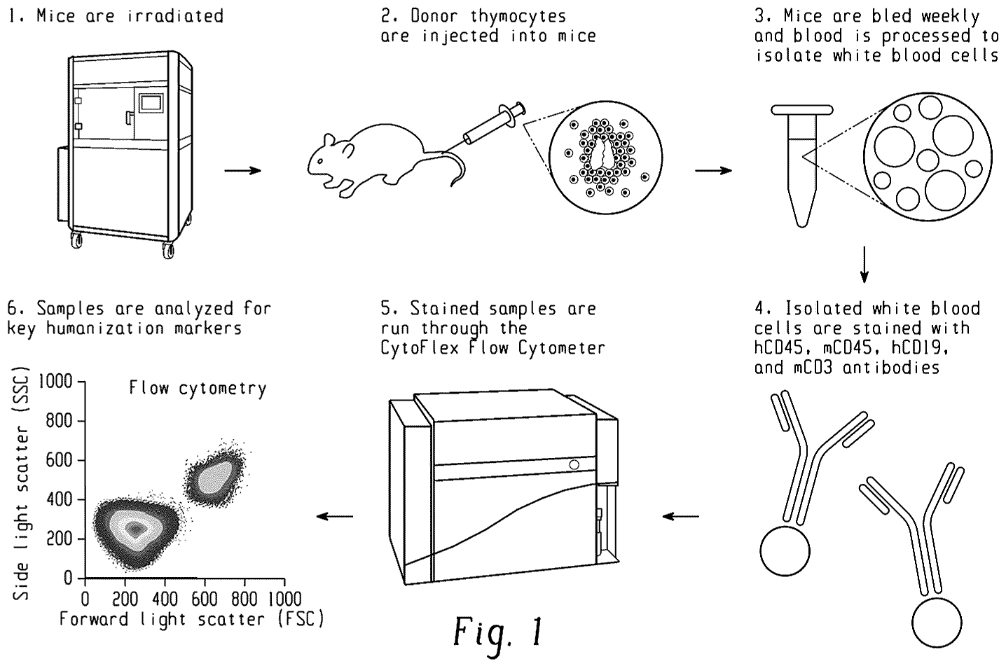

FIG. 1 illustrates an embodiment of the production of a Thymocyte-Hu mouse according to the present disclosure. Briefly, mice are irradiated to provide immune-deficient mice lacking mature T cells, B cells and NK cells. Then donor human thymocytes are injected into the tail of the mice. The mice are bled weekly and blood is processed to isolate white blood cells. The isolated white blood cells can be stained with hCD45 and mCD45 antibodies to determine overall human chimerism, then stained with hCD19 and hCD3 antibodies to determine what types of immune cells are within the human CD45+ population. Typically, these are most/all T cells, but some animals showed some degree of B cells (i.e., hCD19+ cells). T cell markers are analyzed by flow cytometry.

In an aspect, a method of making a humanized animal comprises providing an immune-deficient animal lacking mature animal T cells, B cells and NK cells; and injecting a population of human thymocytes into the immune-deficient animal to provide the humanized animal, wherein the population of human thymocytes are thymocytes collected from human thymus or thymus tissue, and wherein the humanized animal comprises mature human T cells. The humanized animal also optionally comprises mature human B cells.

In an aspect, the animal is not implanted with human thymus tissue, that is, the animal is only injected with a population of human thymocytes.

In an aspect, the animal is a rodent. In another aspect, the animal is a pig.

In an aspect, the mature human T cells in the humanized animal express human CD8 and human CD4 as single-positive (i.e., either CD4⁺ or CD8⁺ but not both at the same time at more than a few %), whereas the injected thymocytes can be single-positive as well as double-positive (i.e., CD4⁺CD8⁺ on the same cell) and double-negative (i.e., not expressing CD4 or CD8).

In an embodiment, the disclosure provides for a method of making a humanized rodent, such as a mouse, rat, squirrel, hamster, porcupine, or beaver. In one embodiment, the method includes providing an immune-deficient mouse lacking mature T cells, B cells, and NK cells.

In another aspect, the rodent is immune-deficient as a result of one or more genetic mutations. In an aspect, the immune-deficient mouse has a mutation in the interleukin-2 (IL-2) receptor γ-chain locus (Il2rg, CD132). The mutation in Il2rg leads to impaired T cell and B cell development, prevents NK development and results in poor lymph node development and organization. Exemplary immune-deficient mouse strains with a mutant Il2rg gene include NSG (NOD.Cg-Prdkc^{scid} Il2rg^{tm1wj1}), NOG (NODShi.Cg-Prdkc^{scid}Il2rg^{tm1Sug}), NRG (NOG.Cg-Rag1^{tm1Mom}Il2rg^{tm1wj1}), BRG (C.Cg-Rag2^{tm1Fwa} Il2rg^{tm1Sug}), BRG (C.Cg-Rag1^{tm1Mom}Il2rg^{tm1Wj1}), BRG (C;129RG; c: 129S4-Rag2^{tm1Flv}Il2rg^{tm1Flv}/), Rag2^{-/-} γc^{-/-} (C;129RG, Stock (H2^{d})-Rag2^{tm1Fwa}Il2rg^{tm1Krf}), B-NDG (NOD.CB17-Prkdc^{scid}IL2rg^{tm1}/BcgenHsd) and the like. The NSG and BRG mice are available from the Jackson Laboratory, the NOG mice are available from Taconic Biosciences, and the B-NDG is available from Inotiv.

In an aspect, the genetically-modified mouse is further myeloablated via irradiation or chemical methods (e.g., busulfan) to remove some of the remaining mouse immune cells/progenitors. Typically, for BLT, NeoTHy and CD34-hu mice, the goal of myeloablation is to make space in the bone marrow for human progenitor cells to engraft and develop. For PBL-hu type mice (including the Thymocyte-Hu), animals are irradiated or chemically treated because it has the effect of increasing the human chimerism/engraftment kinetics. Without being held to theory, it is believed that irradiation sensitizes the animals (i.e., damages the tissues which cause inflammation and other effects that spur the immune response). In an aspect, irradiation may not provide or influence immune deficiency, but rather improve the humanization in the animal.

In an aspect, the rodent is an NSG-SGM3, NOG-EXL or NBSGW mouse. In another aspect, the rodent is an NSG-IL15 or NOG-IL6 mouse.

The method includes injecting a population of human thymocytes into the immune-deficient rodent. Thymocytes can be neonatal, fetal, or adult thymocytes. In an aspect, the population of thymocytes are neonatal thymocytes collected from human neonatal thymus or thymus tissue. Typically, neonatal is defined as the first month of an infant's life. In an aspect, the population of human thymocytes is injected into the tail of the rodent. In another aspect, the population of human thymocytes comprises 5 million or more, preferably 10 million or more human thymocytes. In another aspect, the population of human thymocytes comprises CD3⁺CD4⁺ T cells, CD3⁺CD8⁺ T cells, developing thymocytes (CD4⁺CD8⁺ double positive or CD4⁻CD8⁻ double negative), or a combination thereof.

In an aspect, the population of human thymocytes is from a donor with trisomy 21. DS is a condition in which an extra copy (trisomy) of chromosome 21 is typically obtained by a meiotic error in the gamete. DS occurs in 1 in about 1000 live-births annually. Individuals with DS are prone to blood-related cancers, higher rates of infection, and autoimmunity, but a lower incidence of solid tumors. Trisomy 21 and its gene-dosage effects create significant variability in DS disease severity.

A humanized mouse model of the immune system in DS as described herein would allow the study of personalized pluripotent stem cell (PSC) therapies which may be able to regenerate damaged tissues caused by DS-associated cardiomyopathy and/or immune dysfunction.

The T21 model described herein may be an improvement over the current Ts65Dn model which is unable to fully represent the immunophenotypes of DS. Because of the lack of model systems, little is known about transplantation immune tolerance in DS patients. The T21 Thymocyte-Hu model will allow the assessment of transplant allorejection and tolerance to PSC-derived cells in vivo to better elucidate the complex immunobiology of DS patients and effectiveness of potential treatments.

In an aspect, the humanized rodent is a mouse, and the humanized mouse remains chimeric for more than 40 days post-injection, as determined by percent of human CD45⁺ cells in the mouse blood and tissues. The mice are predominantly engrafted with human (single-positive) T cells, but some animals engraft with a small percentage of human B cells. It may be possible for other human immune cells to engraft in low numbers. The cells circulate in the animals and are present in the spleen and other tissues. This is an important advantage as prior art PBL-Hu mice do not maintain stable engraftment over time. The Thymocyte HU mice persist at moderate levels of engraftment (about 2% human CD45⁺ and remain at that level of engraftment for weeks/months.

In another aspect, the humanized rodent is a mouse, and the humanized mouse survives for 40, 45, 50, 55, 60, 65, 70, 75, 80 or more days or longer post injection, and possesses engrafted human cells throughout the time period. This is an important advantage as prior art PBL-Hu mice both engraft quickly and then die quickly.

Also included here are humanized mice made from the methods described herein. Both the T21 and D21 mouse models described herein will be valuable research tools.

The humanized rodents described herein can be used for vaccine development to determine the mechanisms of antigen-specific T cell responses. Humanized rodents allow the study of human-specific vaccine signatures. The route of vaccine administration can also be optimized in humanized rodents.

Humanized rodents can be used to study the pathogenesis of human infectious agents such as Salmonella species, HIV, HTLV1, EBV, ebola virus, dengue virus, hepatitis B, C, and D viruses, Borellia species, Plasmodium species, cytomegalovirus, and others.

Humanized rodents can also be used to study tumor immunology because they can be engrafted with human cancer cell lines or patient-derived xenografts. Humanized rodents can be used to test the response to cancer treatment and also to assist in the development of novel cancer therapies. Humanized rodents may also have application in personalized oncology.

Humanized rodents can also be used to model autoimmune diseases such as systemic lupus erythematosus, rheumatoid arthritis, and Type 1 diabetes.

Humanized rodents can be used to model metabolic diseases such as nonalcoholic steato-hepatitis (NASH), nonalcoholic fatty liver disease (NAFLD), and others.

Humanized rodents can be used to model patient responses to self and allogenic iPSC therapies as well as to study hematopoiesis.

The invention is further illustrated by the following non-limiting examples.

### EXAMPLES

### Methods

Thymocyte Isolation from Primary Tissue: Neonatal and post-neonatal tissue was collected from the American Family Children's Hospital Department of Pediatric Cardiothoracic Surgery (Madison, WI). Once collected, primary thymus tissue was weighed and photographed. 4 g of thymus tissue was then placed in a petri dish and submerged in 1× Dulbecco's phosphate buffered saline (DPBS) no calcium/no magnesium (DPBS−/−) (Corning) above a bed of ice. The thymus was washed in the petri dish using 1×DPBS−/− enough to submerge most tissue and remove excess blood and debris. Once most blood was removed, the used 1×DPBS (Corning) was discarded. The thymus was immediately resubmerged in fresh 1×DPBS (Corning). Using forceps, the thymus capsule and blood vessels were removed and discarded. Used 1×DPBS−/− (Corning) was then collected and stored in a 50 ml conical tube on ice. Fresh 1×DPBS−/− (Corning) was added, and thymus tissue was fragmented to the size of 1 cm³. Once in desired fragments, 1×DPBS−/− (Corning) was used and added to the previously used 1×DPBS−/− (Corning) in its corresponding 50 ml conical tube. This was done while avoiding disrupting thymus fragments. A new conical tube was acquired, and the cap was removed and replaced with a 100 μm nylon cell strainer (Falcon). Used 1×DPBS−/− (Corning) was filtered through the strainer (Falcon) and any thymus fragments on the strainer were placed back into the petri dish and reserved for other experiments. The trainer was discarded and the cap was refastened.

If the residual 1×DPBS−/− (Corning) solution was pigmented, 7 ml of room temperature Ammonium-Chloride-Potassium (ACK) lysing buffer (Quality Biological) was added. Modifications were made based on 1×DPBS−/− solution saturation levels. If the solution was white and opaque, the ACK lysing step was skipped. After adding ACK lysing buffer (Quality Biological), the solution sat at room temperature for 7 minutes. Afterward, the solution was quenched with 20 ml 1×DPBS−/− (Corning) and centrifuged at 300×g for 10 minutes.

After the solution was centrifuged, the supernatant was aspirated (if the pellet remained pigmented ACK lysing step was repeated). The pellet was then resuspended in fresh 1×DPBS−/− (Corning) up to a volume of 30 ml and a cell count was performed. Once the cell count was completed, the solution was centrifuged at 300×g for 5 minutes. The supernatant was then aspirated, and the pellet composed of isolated thymocytes was resuspended in CryoSto® CS10 (Stem Cell Technologies) at desired volumes and cell counts. 1 ml of CryoSto® CS10 and thymocyte solution was added to cryogenic vials. Cryogenic vials were placed into Corning™ CoolCell™ LX Cell Freezing Container (MilliporeSigma) and stored at −80 C. The following day, the samples were transferred to a nitrogen tank.

Mice Irradiation and Injection: NSG mice (multiple NSG variant strains, including those with transgenic human cytokines e.g., IL15) were irradiated at 200 cGy. After one week 1.5E+07 thymocyte cells were thawed and resuspended in HBSS+5 mL HEPES (Gibco), loaded into 27 G×½ in BD PrecisionGlide™ Needles (BD), and injected via mouse tail.

Blood Draw and Processing for Flow Cytometry: 1.5 ml Eppendorf Tubes® were prepared by adding 200 μl ACD (BD Vacutainer®) per mouse. The mouse was anesthetized using isoflurane and a retro-orbital bleed was performed using a capillary tube. Once the blood was collected, blood from the capillary tube was poured into the Eppendorf Tubes® with ACD (BD Vacutainer®). The Eppendorf Tubes® were then gently shaken and reserved for the antibody addition and lysing procedure.

Blood Draw and Processing for Serum: One 1.5 ml Eppendorf Tube® was set aside per mouse. Mice were anesthetized using isoflurane and a retro-orbital bleed was performed using a capillary tube. Once the blood was collected, blood from the capillary tube was poured into the Eppendorf Tube®.

The serum was then centrifuged at 1000×g for 10 minutes. The blood separated into two distinct layers, the bottom consisted of erythrocytes and the top consisted of serum. The serum was gently extracted avoiding the uptake of erythrocytes. The serum was then dispensed into a new 1.5 ml Eppendorf Tube® and frozen at −80 C.

Flow Cytometry Antibody Preparation: Flow Cytometry Staining Buffer (FAC's) buffer was made using 500 ml of PBS+10 ml of Fresh Bovine Serum (FBS) and then filtered.

To measure mouse chimerism during weekly blood draws the following antibodies were used: APC Mouse Anti-Human CD45 (BD Pharmigen™), PE-Cy 5 Mouse Anti-Human CD19 (BD Pharmigen™), PE Mouse Anti-Human CD3 (BD Pharmigen™), FITC Rat Anti-Mouse CD45 (BD Pharmigen™). A master mix was made using 5 μl of APC Mouse Anti-Human CD45 (BD Pharmigen™), 5 μl PE-Cy 5 Mouse Anti-Human CD19 (BD Pharmigen™), 5 μl PE Mouse Anti-Human CD3 (BD Pharmigen™), and 1 μl FITC Rat Anti-Mouse CD45 (BD Pharmigen™) per number of samples. FACS was added so that there was a total of 100 μl of total volume per sample.

To conduct a more extensive T-cell panel the following antibodies were used FITC Mouse Anti-Human CD45 (BD Pharmigen™), PE Mouse Anti-Human CD45RA (BioLegend®), PE-CF594 Mouse Anti-Human CD25 (BioLegend®), PerCP-Cy5.5 Mouse Anti-Human CCR7 (BioLegend®), PE-Vio770 Anti-Human CD45RO (Miltenyi Biotec), APC Mouse Anti-Human CD62L (BD Pharmigen™), CD8 APC-R700, APC-Cy7 Mouse Anti-Human CD31, BV421 Mouse Anti-Human CD3 (BD Biosciences), BV510 Mouse Anti-Human CD4 (BioLegend®), and CD127 BV605. A master mix was made using 2 μl FITC Mouse Anti-Human CD45 (BD Pharmigen™), 0.5 μl PE Mouse Anti-Human CD45RA (BioLegend®), 1.25 μl Mouse Anti-Human CD25 (BioLegend®), 2 μl PerCP-Cy5.5 Mouse Anti-Human CCR7 (BioLegend®), 0.5 μl PE-Vio770 Anti-Human CD45RO (Miltenyi Biotec), 5 μl APC Mouse Anti-Human CD62L (BD Pharmigen™), 1 μl CD8 APC-R700, 3 μl APC-Cy7 Mouse Anti-Human CD31, 0.6 μl BV421 Mouse Anti-Human CD3 (BD Biosciences), 1.5 μl BV510 Mouse Anti-Human CD4 (BioLegend®), and 4 μl CD127 BV605 per number of samples. FACs buffer was added to the master mix so that there was a total of 50 μl of total volume per sample.

Antibody Addition and Lysing Procedure: Before conducting the lysing procedure, the following reagents were made: 1× lysing buffer and Flow Cytometry Staining Buffer (FACs). 1× lysing buffer was prepared using 45 ml of deionized water and 5 ml of lysing solution. FAC's buffer was made using 500 ml of PBS+10 ml of Fresh Bovine Serum (FBS) and then filtered.

For the extensive T cell panel experiments 50 μl of the corresponding master mix was used per sample, while 100 μl of the designated chimerism master mix was used for weekly chimerism testing. Once appropriate antibodies were added, the samples were incubated in the dark for 15 minutes at room temperature.

When the incubation period was over 1 ml of 1× lysing solution was added per sample, ensuring to add solution to no more than two samples at a time. Tubes were immediately vortexed after adding 1× lysing solution. Samples were incubated at 37 C, protected from light, for 3 minutes. After, incubated samples were centrifuged at 300×g for 5 minutes. Once finished supernatants were disposed of ensuring to keep 100 μl of total volume per sample. Samples were then vortexed to break up the pellet. 1 ml of FACs buffer was added to each sample, vortexed, and then centrifuged at 300×g for 5 minutes. After this step was completed, the supernatant was disposed of ensuring that 100 μl total volume remained in the samples. The pellet was vortexed, and the steps post-addition of antibodies were repeated. Flow cytometry was then performed on a CytoFLEX Flow Cytometer (Beckman Coulter) and analyzed using FlowJo Version 10.9.0 software.

CM Differentiation: PSC-derived cardiomyocytes (CMs) were differentiated from the WC-24-02-DS-B/WB68072 line implementing the Alternate GiWi protocol, using small molecule temporal modulation of the Wnt pathway, as previously described in the art. Before differentiation, PSCs were maintained in TeSR™-E8™ media (StemCell Technologies WiCell Formulation) on Matrigel®-coated (Corning) cell culture plates until they reached full confluency.

Flow for Purity CMs: Following 17 days of differentiation, cardiomyocytes (CMs) cultured on Matrigel®-coated (Corning) cell culture plates were harvested and stained for Anti-Cardiac Troponin T (Clone 1C11) Antibody (Abcam) and Goat anti-Mouse IgG (H+L) Cross-Adsorbed Secondary Antibody Alexa Fluor 647 (ThermoFisher Scientific) to assess CM purity as described in the art. Flow cytometry was performed on a CytoFLEX Flow Cytometer (Beckman Coulter) and analyzed using FlowJo Version 10.9.0 software.

### Example 1: Creation of Humanized Mice with a Trisomy 21 [T21] Genotype

A schematic of the method is shown in FIG. 1. Three human donor groups were used to create humanized mice: 1) Thymocyte donors with a T21 genotype, 2) thymocyte donors without the T21 genotype (euploid, D21), and 3) peripheral blood mononuclear donors (PBL-Hu). NSG mice irradiated at 200 cGy and NSG-IL 15 mice irradiated at 100 cGY were used. A mosaic DS iPSC line (D21) was differentiated into cardiomyocytes using GiWi monolayer protocol.

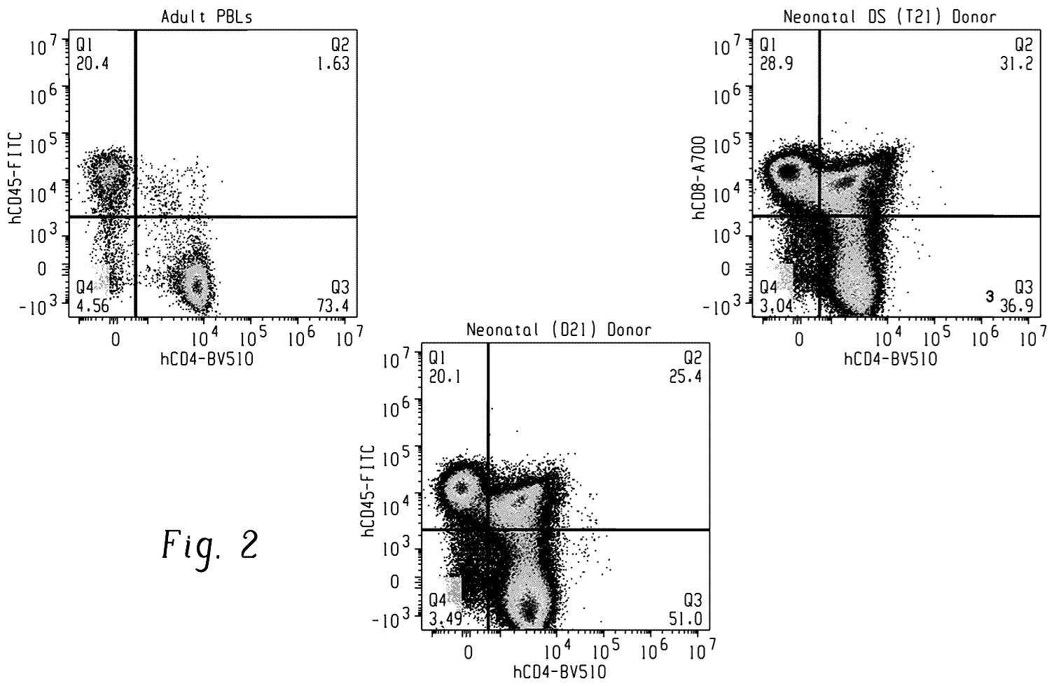

FIG. 2 shows the input thymocyte CD4 and CD8 profile. Adult peripheral blood leukocytes (PDLs, left), neonatal thymus-derived thymocytes from euploid donors (D21, center), and Down Syndrome (DS) donors with T21 (T21, right), were assessed for CD3⁺CD4⁺ and CD3⁺CD8⁺ T cells and/or developing thymocytes prior to injection into the mice.

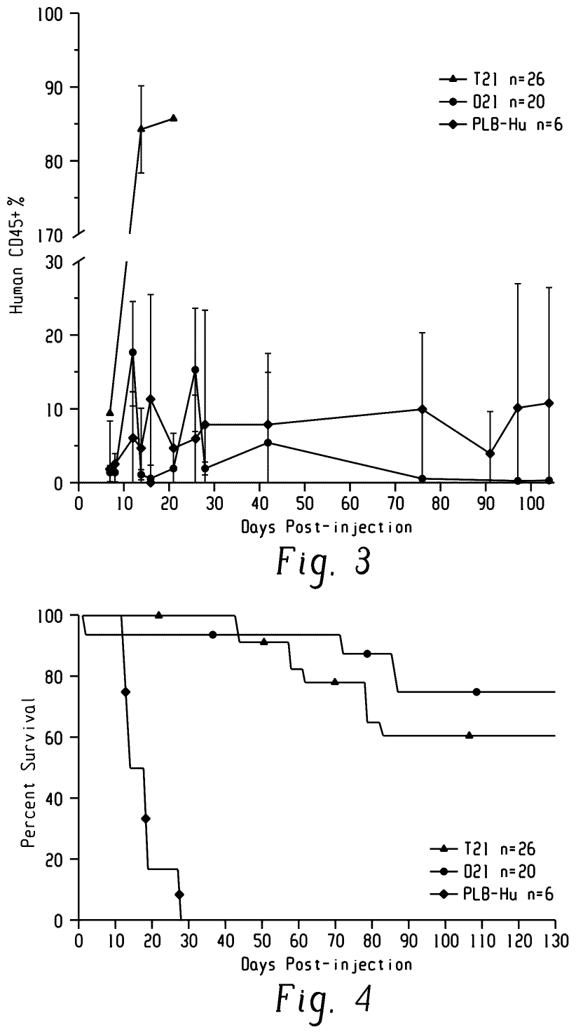

FIG. 3 illustrates the human chimerism of the mice over time. The percent human CD45 versus days post-injection in T21 and D21 donors is shown. T21 donors were able to maintain chimerism the longest, while both D21 and T21 were both chimeric much longer than PBL-HU controls which all died within 30 days post-injection.

FIG. 4 illustrates the survival of the T21 and D21 mice post thymocyte injection. D21 and T21 exhibited similar survival, 80% or more of the mice survived past 80 days. PBL-HU controls all died within 30 days post-injection.

### Example 2: Characterization of Thymocyte-Humanized D21 and T21 Mice

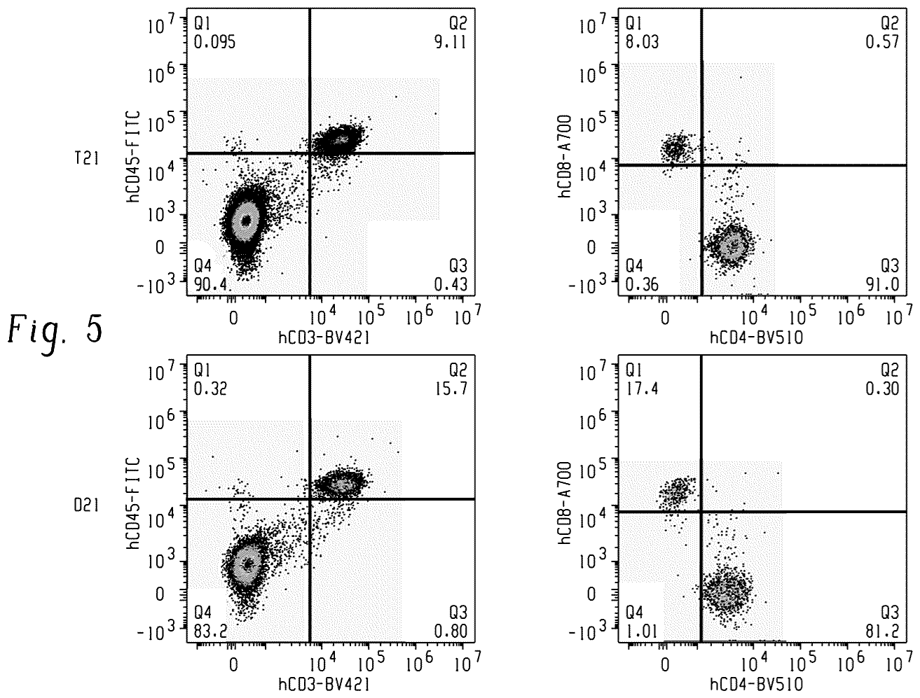

FIG. 5 shows the characterization of the D21 and T21 thymocyte humanized mice. Human (h) CD45⁺CD3⁺ cells show typical levels of hCD8⁺ and hCD4⁺ in flow cytometry, and no significant double positive hCD4⁺CD8⁺ cells. When mice are injected with single, double positive, and double negative thymocytes, only the single positive ones are what are in circulation in the animals. Without being held to theory, it is believed that the injected double positive cells simply die, or mature into single positive cells.

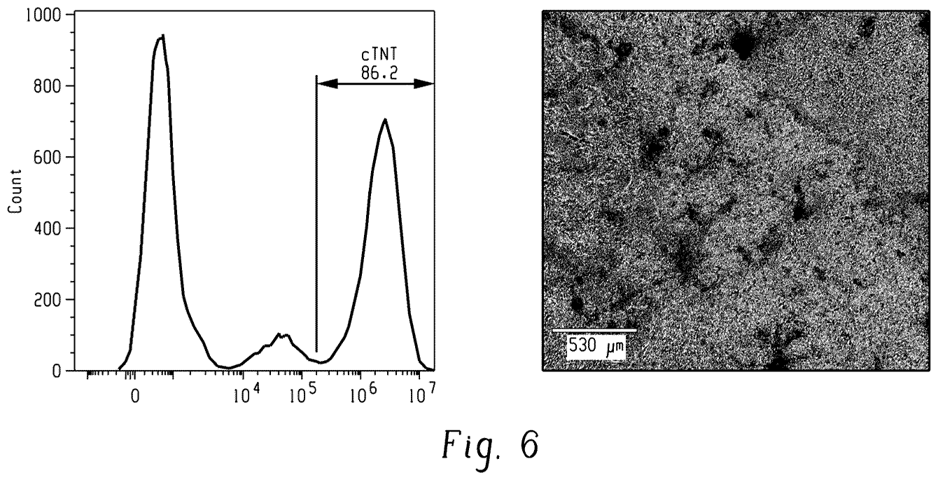

FIG. 6 shows the DS mosaic iPSC line was differentiated into cardiomyocytes. WC-24-02-DS-B/WB68072 line (D21 from DS patient) was differentiated into 86.2% cTNT⁺ cardiomyocytes via GiWi method.

### CONCLUSIONS

As shown herein, D21 and T21 thymocyte-humanized mice both maintained chimerism and survived significantly longer than PBL-Hu mice. The T21 thymocyte-humanized mice were able to maintain chimerism longer than PBL-Hu. While hCD4⁺CD8⁺ cells were present on injection, minimal hCD4⁺CD8⁺ cells were present in the D21 and T21 mice. Also, graft versus host disease is significantly diminished in D21 and T21 thymocyte-humanized mice compared to PBL-Hu mice.

Advantageously, the thymocytes used to prepare the thymocyte-humanized mice can take advantage of cells that are typically discarded as the thymus is typically discarded during neonatal heart surgery. Also, there are abundant thymocytes remaining after the processing of thymus tissue during the preparation of Neo-Thy mice. Further preliminary results show B-cell engraftment in the thymocyte-humanized mice which is rare in adult PBL-Hu mice.

Advantageously, the Thymocyte-Hu mice are characterized by human blood cells in the circulation, similar to PBL-Hu mice. The thymocyte-humanized mice have utility in studies of human T cell development and function.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of making a humanized animal, comprising
providing an immune-deficient animal lacking mature animal T cells, B cells, and NK cells; and
injecting a population of human thymocytes into the immune-deficient animal to provide the humanized animal;
wherein the population of thymocytes are thymocytes collected from human thymus or thymus tissue; and
wherein the humanized animal comprises mature human T cells expressing human CD8 and human CD4.

2. The method of claim 1, wherein the population of human thymocytes comprises neonatal thymocytes.

3. The method of claim 1, wherein the population of human thymocytes comprises 10 million or more thymocytes.

4. The method of claim 1, wherein the population of human thymocytes comprises CD3⁺CD4⁺ T cells, CD3⁺CD8⁺ T cells, developing human thymocytes, or a combination thereof.

5. The method of claim 3, wherein the animal is a rodent.

6. The method of claim 5, wherein the rodent is immune-deficient as a result of one or more genetic mutations.

7. The method of claim 5, wherein the rodent is an NSG-SGM3, NOG-EXL or NBSGW mouse.

8. The method of claim 5, wherein the rodent is an NSG-IL15 or NOG-IL6 mouse.

9. The method of claim 5, wherein the population of human thymocytes is injected into the tail of the mouse.

10. The method of claim 1, wherein the animal is a pig.

11. The method of claim 1, wherein the population of human thymocytes is from a donor with trisomy 21.

12. The method of claim 1, wherein the humanized animal is a mouse, and the humanized mouse remains chimeric for more than 40 days post-injection, as determined by percent of CD45⁺.

13. The method of claim 1, wherein the humanized animal is a mouse, and the humanized mouse survives for 40 days or longer post injection, and possesses engrafted human T cells throughout the post-injection time period.

14. A humanized animal prepared by the method of claim 1.

15. The humanized animal of claim 14, wherein the population of human thymocytes is from a donor with trisomy 21.

16. The humanized animal of claim 15, wherein the animal is a mouse.
